(19) United States
(12) Patent Application Publication
(10) Pub. No.: US 2006/0051295 A1
(43) Pub. Date: Mar. 9, 2006

(54) Estrogen receptor interaction with a transcription factor

(76) Inventors: Christine J. C. Boersma, Oss (NL); Alain J. Van Gool, Oss (NL)

Correspondence Address: INTERVET INC.;PATENT DEPARTMENT, PO BOX 318, MILLSBORO, DE, 19966-0318, US

(21) Appl. No.: 10/505,822

(22) PCT Filed: Feb. 21, 2003
(86) PCT No.: PCT/EP03/50027

§ 371 Date: Jul. 12, 2005

(30) Foreign Application Priority Data
Feb. 25, 2002 (EP) 02075789.4

(51) Int. Cl. A61K 49/00; G01N 33/567

(52) U.S. Cl. 424/9.2; 435/7.2

(57) ABSTRACT

The invention provides a method to measure a direct interaction between an estrogen receptor and a transactivation protein in an in vitro system by providing for a detection parameter proportionally related to the degree of the interaction, whereby the transactivation protein is c-Rel. In an embodiment of the invention a yeast two-hybrid assay for the measurement is provided. Also is provided a method to influence the direct interaction between an estrogen receptor (ER) and c-Rel in a system, by addition to the system of a compound, which influences the direct interaction between an estrogen receptor (ER) and c-Rel.

The invention relates to a method to measure a direct interaction between an estrogen receptor (ER) and a transactivation protein in an in vitro system by providing for a detection parameter proportionally related to the degree of the interaction.

Several important medicinal compounds have their therapeutic effects in an organism by an influence on transcription factors. In particular, this mechanism of action is used by those medicines, which act by mimicking steroidal hormonal actions, such as certain contraceptives and anti-inflammatory compounds. Transcription factors are proteins controlling the read-out of genetic DNA material in the cells of an organism. It is this mechanism of control which determines many properties of a living cell. The control of expression of genes in the cells can be permanent or can be transient. Transient changes are changes in response to changing environments of the cell. The presence of other cell surfaces and other molecules in the environment of the cell are a source of signals to a cell and lead to a change of its functioning. The transcription factors play an essential role as mediators of external influences on the cell. In this sense, transcription factors are part of signaling mechanisms.

Well known transcription factors are nuclear receptors, such as receptors for the steroid hormones estradiol, progesterone, cortisol etc. Another transcription factor family is referred to as the nuclear factor κB (NF-κB) transcription factor family. Transcription factors of the NF-κB family are dimers composed of two subunits out of the group RelA(=p65), RelB, c-Rel, P50 and P52. A signaling system, based on mobilisation of NF-κB, operates by responding to cytokine molecules from outside the cell via an inhibiting protein IκB. The inhibitor of NF-κB, IκB, forms inactivating complexes with NF-κB, but these complexes are susceptible to enzymatic removal of IκB by phosphorylations in response to cellular exposure to cytokines. The enzymatic removal of this inhibitory factor from I-κB/NF-κB complexes make NF-κB available for activity on the genome. Another distinct transcription factor family, c/EBP-β, will be mentioned later in this description.

Transcription factors operate as complexes comprising multiple subunits. These complexes have affinity for a domain in the genome, which domain is called a response element. One or more response elements in a DNA chain form a location from which the bound transcription factor interacts with a larger portion of the genome, the promotor region. The complexation of the transcription factor with response elements and a promotor region in DNA determine the rate of transcription of the gene linked downstream to the promotor on the DNA. It is striking that many transcription factors are binding to DNA as dimers. Thus, the nuclear steroid hormone receptors are usually composed of two identical or closely related subunits. In modern biochemical theories, the notion that transcription factors belong to separate classes without mutual interaction is now obsolete and replaced by the notion that transcription factors interact across classes of transcription factors. Thus, selective binding interactions were demonstrated between the glucocorticoid steroid receptor monomer (GR) with RelA (Harnish, Endocrinol. 141: 3403-3411, 2000). High affinity binding was discovered between androgen receptor (AR) with RelA (Panet-Raymond et al., Mol Cell Endocrin., 167, pp 139-150, 2000). The estrogen receptor a monomer is shown to form complexes with c-Jun (Teyssier et al, J Biol Chem 276 pp 36361-36369, 2001). It is believed that such complexes are building blocks of complexes which function as transcription factors or, alternatively, absorb an amount of an available transcription factor, thereby encapsulating the factor into an inactive complex (Ray and Prefontaine Proc. Natl. Acad. Sci. 91: 752-756, 1994; Kalkhoven et al. J. Biol. Chem. 271: 6217-6224, 1996)

Since the signaling mechanisms have mutual influences owing to above-mentioned direct protein-protein interactions between the transcription factor subunits, the terms cross-talk and transrepression are used to describe those mutual influences. Transrepression refers to the negative influence of one signaling pathway on another. The present invention more specifically focusses on the interactions between a member of the estrogen receptor family with an NF-κB protein and the use of the interaction for manufacture of medicines.

Several studies exist that show efficient repression of NF-κB signaling by ER (Quaedackers et al. Endocrinology 142:1156-1166, 2001). The mechanism underlying the transactivating and transrepressing effects of ER on NF-κB signaling is not clear. There is a possibility that the nuclear receptor and NF-κB may associate into large multimeric complexes. Such complexes can contain nuclear receptor, NF-κB and several other nuclear (co-)factors and may still be capable to bind the DNA. An example of a concept of reciprocal interaction between a nuclear receptor and an NF-κB signaling pathway is depicted in FIG. 1

[image]

(FIG. 1) Schematic representation of reciprocal transrepression by ER and NF-κB proteins. For other nuclear receptors, such as GR and PPARγ, similar kinds of functional interactions with NF-κB have been described. It is at present not yet clear whether formed complexes of NF-κB with nuclear receptors consists of monodimers or dimers.

The present invention provides for a method to measure a direct interaction between an estrogen receptor (ER) and a transactivation protein in an in vitro system by providing for a detection parameter proportionally related to the degree of the interaction, characterised in that the transactivation protein is c-Rel.

Thus far, only weak protein-protein interaction between ER and certain types of NF-κB was reported (Stein and Yang, Mol. Cell. Biol. 15: 4971-9, 1995; Speir. Circ. Res. 87: 1006-1011, 2000) and this interaction was independent from the presence of an ER ligand. In contrast, the interaction between estrogen receptor and c-Rel described here is dependent on the presence of a compound that binds to the estrogen receptor and by doing so can induce the interaction between the estrogen receptor and c-Rel.

It is useful to measure the interaction between an estrogen receptor (ER) and c-Rel, and also the influence of a compound thereon since ER/NF-κB interactions are involved in certain effects of estrogens on the cardiovascular system, bone and the central nervous system (CNS). These are three major tissues where estrogens display beneficial effects in medical treatments. An additional role of ER/NF-κB interactions in progression of breast cancer has been suggested (Rodriguez et al. Immunogenetics 39: 161-167, 1994; Nakshatri et al. Mol. Cell. Biol. 17: 3629-3639, 1997), indicating a role of NF-κB inducible genes in malignancy and chemotherapeutic resistance.

Although knock-out phenotypes do not show clear functions associated to one of the NF-κB types only (Attar et al., Cancer Biol 8: 93-101, 1997), the absence of both p50 and p52 in double knock-out mice results in osteopetrosis, supporting the role of NF-K in bone metabolism (Iotsova et al., Nat. Med. 3: 1285-1289, 1997).

In bone, NF-κB proteins play an important role in modulation of inflammatory genes (e.g. cytokines) and cell adhesion molecules. Both types of modulatory molecules are important teamplayers in bone homeostasis. In particular cytokines, such as Il-1β, IL-6 and TNFα, stimulate osteoclast precursors and mature osteoclasts and thus enhance bone resorption (Manolagas, Bone 17: 63S-67S, 1995). Estrogens contribute significantly to the maintainance of bone mass by inhibiting the production of such cytokines (Ray and Prefontaine Proc. Natl. Acad. Sci. 91: 752-756, 1994; Stein and Yang, Mol. Cell. Biol. 15: 4971-9, 1995]. The importance of NF-κB in bone metabolism is further exemplified by knock-out studies as mentioned above (Iotsova et al., Nat. Med. 3: 1285-1289, 1997). In the CNS, several reports indicate an involvement of NF-κB in various processes but its exact contribution is still unclear. Factors that are linked to synaptic plasticity, long-term memory and Alzheimer disease, have been identified as brain-specific activators of NF-ΛB (O'Neill and Karltschmidt, Trends Neurosci. 20: 252-258, 1997; Grilli et al, J. Biol. Chem. 271: 15002-15007, 1996). In addition, inhibition of NF-κB is associated with survival of neuronal cells (Maggirwar et al. J. Neurochem. 74: 527-539, 2000, Irving et al., Neurosci. Lett. 288: 45-48, 2000). Additional associations have been made between NR/NF-κB cross-talk and the serotonin system (Wissink et al, Mol Endocrinol. 15(4):543-52, 2001), the latter being a valid potential entry for treatment of depression. In CV, estrogens are thought to inhibit the development of atherosclerosis. One of the earliest events in the development of atherosclerosis is the infiltration of monocytic cells into the vessel wall. A critical step is the recruitment of monocytes from the circulation. This recruitment involves several steps. The initial interaction between monocyte and endothelium appears to be transient, resulting in the rolling of monocytes along the vessel wall. The rolling monocytes then become activated by locally secreted factors generated by the endothelium, resulting in their arrest and firm adhesion to the vessel wall. Finally, the monocytes transmigrate the endothelium. The initial rolling interactions are mediated by the selectins (e.g. E-selectin), whereas firm adhesion and diapedesis appear to be mediated by the interaction of integrins on the surface of monocytes with immunoglobulin gene superfamily members expressed by endothelial cells (e.g. ICAM, VCAM). Expression of E-selectin, VCAM and ICAM is enhanced as a result of transcriptional activation of all three genes by cytokines via NF-κB signaling. Therefore, by interfering with this NF-κB induced expression of cell adhesion molecules, the infiltration of monocytes resulting in atherosclerosis, may be reduced. Expression of c-Rel in the blood vessel wall is clearly enhanced in response to ballooning (Landry et al., Am. J. Pathol. 151: 1085-1095, 1997), suggesting a role for c-Rel in vascular transformation.

Other interesting functions of c-Rel are its function in control of programmed cell death and its role in activation of macrophages. Overexpression of c-Rel induces apoptosis in bone marrow cells, whereas it is protective against cell death in fibroblasts (Abbadie et al., Cell 75: 899-912, 1993). Macrophages from mice that are deficient for c-Rel produce higher than normal levels of inflammatory cytokines (Grigoriadis et al., EMBO J. 15: 7099-7107, 1996).

The concept that c-Rel seems more crucial than RelA for ER/NF-κB transrepression is new, regarding information as present in literature. Therefore, it offers a unique opportunity for selection of compounds that selectively induce ER/c-Rel association. The assays to identify such compounds are hereby provided. A specific embodiment of the invention is a yeast two-hybrid assay.

With such methods according to the invention there is also provided a method for selection, out of a number of compounds, of a compound for therapeutic efficacy in osteoporosis by carrying out the method, according to the invention as described above, repeatedly, optionally in parallel, in the presence of each of the number of compounds in the system and selecting a compound which enhances the interaction between an estrogen receptor and c-Rel.

Further improvement of the selection of a compound which enhances the interaction between an estrogen receptor and c-Rel can be done in animal models, preferably an animal model for osteoporosis.

Animal models include a method which monitors the capacity to prevent bone loss or stimulate bone formation in an in vivo model for osteoporosis. In this model, three month-old ovariectomised rats treated for four weeks orally with the compound to be tested, as described by Ederveen. Journal of Bone & Mineral Research. 14(11):1963-70, 1999]. Immediately after ovariectomy a four week oral treatment with various doses of the test compound is started. After four weeks of treatment trabecular bone mineral density (TBMD in mg/cm3) of the distal metaphysis of the femur is measured by pQCT (peripheral Quantitative Computed Tomography machine; XCT 960A, Stratec, Birkenfeld, Germany). A scan including a 1 mm thick slice, with a resolution of 0.148×0.148 mm, is taken 5 mm from the distal end of the femur. Intra- and inter-assay variation for the measurement of trabecular bone density in the distal femur is 3%. The XCT-960A is calibrated with a standard of hydroxyapatite embedded in acrylic plastic. In addition, cortical bone mineral density can be measured at the medial site of the femur.

The provision of compounds which induce the direct interaction between an estrogen receptor and c-Rel in a system, provide a method to influence the interaction in a system by adding a compound to the system, which compound influences the direct interaction between an estrogen receptor (ER) and c-Rel. In a more specific embodiment of this aspect of the invention, the method to influence the interaction in a system by adding a command to the system is obtained by selecting an in vitro culture medium as the system. Most preferred is to use these methods to influence the interaction between an estrogen receptor and c-Rel by addition of a compound to the system with a compound, which influences the interaction selectively in comparison to an influence of the compound on ER transcriptional activity.

Compounds which are suitable for the methods defined in the previous paragraphs can be used for the manufacture of a pharmaceutical composition for use in a therapeutic treatment to influence the interaction between estrogen receptor and c-Rel. Such a therapeutic treatment is for example, and preferably, a treatment for osteoporosis.

In this description the terms have the following meaning: A direct interaction between two proteins is meant to be a physical contact based on chemical affinity under circumstances comparable to the natural environment of the proteins. However, this can not only mean an increase or a decrease in the binding between these proteins, but also a modification of the secondary structure of the binding complex, making it more or less efficacious in preventing or inducing transcriptional activity.

Transactivation protein, protein in general called transcription factor that has a transactivating activity on gene expression. Transactivation proteins interact with co-factors (co-activators, co-repressors, others) and other (transcription) factors that belong to the transcription machinery and with which they together form a complex that modulates transcription initiation.

estrogen receptor (ER) transcriptional activity is the transactivating activity of the estrogen receptor obtained by direct interaction of the activated ER dimer with specific ER responsive regulatory elements (ERE) gene regulatory parts of the genome. As a consequence of activation of the ER dimer it interacts with a specific set of co-activators and other cofactors, (transcription) factors and in complex with the transcription machinery activate transcription initiation. By “regulatory element” or “promoter” is meant a DNA sequence that is capable of binding directly or indirectly to RNA polymerase in a cell and initiating transcription of a downstream (3′ direction) coding sequence. A promoter may be linked to a heterologous reporter gene capable of signaling the activation of the regulatory element. In such a construct, the promoter influences the transcription from the heterologous gene. Suitable reporter genes are for instance luciferase, chloramphenicol acetyl transferase, beta galactosidase and secreted placental alkaline phosphatase.

An estrogen receptor (ER) is a protein that binds the natural hormone 17β-estradiol and belongs to the family of nuclear receptors and more specific to the subfamily of steroid receptors. The nomenclature of nuclear receptors has been described (Laudet V., Auwerx J., Gustafsson, J.-A. & Wahli, W. (1999) Cell 97 161-163). At present two closely related estrogen receptors are known, being ERα and ERβ. Also included are those variant and modified proteins that bind 17β-estradiol and are functional equivalents of the estrogen receptor and that retain the described biological function in its interaction with c-Rel. With a “functional equivalent” is to be understood a molecule capable of excerting the same biological function as the molecule it refers to, having major structural features in common and being available as an alternative means to the skilled person on the basis of generally available technology in this field. Often, such equivalents are defined to be proteins with 90%, 95%, 98% or 99% aminoacid identity to the proteins defined by sequence data. Clearly such definitions are comprised as more specific definitions in the present definition, provided the function of the estrogen receptor equivalent in binding to c-Rel or vice versa, the c-Rel equivalent to the estrogen receptor is retained. Percentage identity is hereby defined as:

$$Identity ( \% ) = \frac{Number of identical residues between two sequences}{Length of aligned sequences - Length of all gaps}$$

after optimal alignment of the sequences. Optimal alignment can be performed using the algorithm of Needleman and Wunsch (J. Mol. Biol. 48; 443-453 (1970)) that maximizes the number of matches and minimizes the number of gaps.

Other receptors that are closely related to the estrogen receptors are the ER related receptors (ERR) ERRα, ERRβ and ERRγ, but these are not included within the presently used definition of an estrogen receptor, in view of the lack of demonstrated ligand binding with ERRs.

The estrogen receptor-α (ERα) is the protein coding for the a-type of estrogen receptor, being the translation product of the ERα gene. It is the preferred protein for use in the methods according to the invention.

c-Rel is the protein product from the c-Rel gene. c-Rel is one of the members of the NF-κB family that share a homology in the Rel domain and are involved in gene regulation (Liou and Baltimore, Current Opinion in Cell Biology, 5: 477-487, 1993) including but not limited to NF-κB1, Lyt-10, c-Rel, RelA and RelB. Also included are other functional equivalents of c-Rel that retain its biological function in the described interaction with an estrogen receptor, and which are proteins that the c-Rel gene encodes for, and that are the products of transcription and translation of the c-Rel gene.

An in vitro system is a culture medium in which cells are cultured and which are optimized for optimal growth and/or for optimal responses in the read-out system it is used for. Typical examples of medium for the cell culture are DMEM, α-MEM , RPMI or DF. These media are often but not necessarily supplemented with additional factors including growth factors, serum, vitamins, aminoacids and antibiotics. In the in vitro systems described, 17β-estradiol or test compounds are added to the medium in order to test the effects of the test compounds on ER-cRel interactions.

A detection parameter is the method used to measure the interaction between estrogen receptor and c-Rel. This includes the use of promoter reporter constructs that contain NF-κB or ER responsive regulatory elements fused to a heterologous reporter gene capable of signaling the activation of the regulatory element. In addition, direct interaction can be measured by using chimerae proteins of ER and c-Rel with either the transactivating or DNA binding part of the β-gal (as described for yeast 2-hybrid), by which direct interaction between ER and c-Rel can be measured as induction of a gal reporter construct. Also other promoter derived elements can be used in combination with a reporter gene, of which activation of the reporter gene visualizes the desired interaction between the estrogen receptor and c-Rel. This may also be a gene of which the expression is modulated by c-Rel or estrogen receptor or the protein product of such a gene, such as is exemplified for Il-6 or other cytokines. More specifically, the parameter may even refer to the in vitro biological effect of the interaction between the estrogen receptor and c-Rel, if it is first established that it specifically relates to the interaction.

Enhancing the interaction by a compound means enhancing by increasing the affinity between the estrogen receptor and c-Rel or enhancing the effects of the complex in which an estrogen receptor is being bound to c-Rel. Without being bound by theory in the definition of this invention, it is thought that an enhancing compound changes the secondary structure of the estrogen receptor and this changed secondary structure has the proper conformation to enter into an effective complex with c-Rel.

Influencing the interaction selectively in comparison to an influence of the compound on ER transcriptional activity means that a compound which influences the interaction does so, depending on the selected system, at dosage levels or concentrations, half or less than those at which ER transcriptional activity is observed.

A yeast two-hybrid assay is a cellular method to quantify the degree of interaction between two proteins. This is done by producing constructs that encode for fusion proteins of the two proteins to be studied, in this case ER and c-Rel, with either the transactivating or DNA binding part of the Gal4. The transactivating and DNA binding part of gal are separately not capable of inducing reporter activity of a gal4-responsive β-galactosidase reporter construct. Due to the interaction of the two proteins the transactivating and DNA binding parts of Gal4 will come in close proximity and as a result induction of a gal-responsive reporter will occur. More specificcally, in this embodiment of the invention the estrogen receptor is fused to the DNA binding domain (DBD) of Gal4, whereas c-Rel is fused to the activation domain (AD) of Gal4. The ER/Gal4-DBD fusion protein will bind to Gal4 response elements that are upstream of the reporter gene β-galactosidase in the yeast strain. Interaction between ER and c-Rel is compound dependent, therefore increasing concentrations of compound in the in vitro system will increase the number of ER and cRel molecules that interact, that can be measured as an increase of β-galactosidase reporter activity until saturation levels are reached. The amount of β-galactosidase reporter activity therefore is a measure for the degree of interaction between estrogen receptor and c-Rel.

Degree of interaction refers to the number of ER and cRel molecules that interact. Similarly to yeast two-hybrid, in transfection studies or other systems the degree of interactions can be measured by a specific detection parameter that is dependent on the number of estrogen receptor and cRel molecules that interact. The number of estrogen receptor and cRel molecules that interact are therefore proportionally related to the detection parameter used.

Selection, out of a number of compounds, of a compound for therapeutic efficacy is commonly used in pharmaceutical industry and affiliated industries to find compounds for medicinal use. Large stocks of compounds are screened for active compounds and further optimalisation is obtained by directed synthesis of the most effective compounds. The method provided by this invention is suitable for screening large numbers of compounds in search for an active compound which enhances the interaction between an estrogen receptor and c-Rel. In such screening programs the method of measuring the interaction is performed repeatedly, often for many compounds in parallel, in order to obtain comparative data, and on the basis of the comparative data, select the most active compounds for development as medicinal compounds.

A therapeutic treatment to influence the interaction between an estrogen receptor and c-Rel is a treatment with the purpose to influence the interaction in order to increase or decrease the interaction in the body of an animal or human person. Such treatment is aimed at obtaining the indicated biochemical effect because in modern medicine treatments are usually defined on the basis of the intended biochemical effect rather than on the basis of the therapeutic effect to be obtained. The latter, should of course be the intended and necessary result of the treatment. A specific example of a treatment to influence the interaction between an estrogen receptor and c-Rel is a better treatment of osteoporosis. The biochemical mechanisms in osteoclasts and osteoblasts is such that a compound aimed at the interaction, subject of this invention, is effective in treating osteoporosis with less other nonintended effects. However, by providing the methods to influence, preferably selectively, the interaction between an estrogen receptor and c-Rel, the invention makes a broader contribution to the art of drug treatment of diseases than only a treatment for osteoporosis. The relevance for other therapeutic areas has been explained elsewhere in this description.

The term ‘system’ refers to a molecularly and/or biologically defined environment and refers to in vitro systems as well as to in vivo systems in which a compound can be introduced. In vitro systems are mixtures containing both ER and c-Rel proteins, either in intact cells or in homogenised biochemical compositions, and in vivo systems are cells containing both ER and c-Rel proteins, which cells are within a human or animal body.

Compounds which influence the direct interaction between an estrogen receptor and c-Rel are provided in the examples.

Such compounds can be added directly to the system of by way of a pharmaceutical composition.

A method of preparation of a pharmaceutical composition by mixing the agent with one or more pharmaceutically acceptable auxiliaries is well known in the art (Gennaro et al., Remmington: The science and practice of pharmacy; (20th ed., Lippincott Williams & Wilkins, Baltimore, Philadelphia, 2000, see especially Part 5: Pharmaceutical manufacturing). Suitable auxiliaries are described in e.g. the Handbook of Pharmaceutical Excipients (2^{nd} Edition, Editors A. Wade and P. J. Weller; American Pharmaceutical Association; Washington; The Pharmaceutical Press; London, 1994). The mixture of the agent and the pharmaceutically acceptable auxiliary may be compressed into solid dosage units, such as pills, tablets, or be processed into capsules or suppositories. By means of pharmaceutically suitable liquids the agent can also be applied as an injection preparation in the form of a solution, suspension, emulsion, or as a spray, e.g. nasal spray. For making dosage units, e.g. tablets, the use of conventional additives such as fillers, colorants, polymeric binders and the like is contemplated.

### EXAMPLES

### Example 1

### Modulation of Adhesion Molecules by NF-κB and its Repression by Estrogens

This example focusses on promoters of cell adhesion molecules, such as E-selectin, ICAM and VCAM, their regulation by NF-κB proteins and their subsequent repression by a compound inducing ER-cRel interaction. For studying repression of NF-κB signaling via ERα, we used the promoters of the genes encoding three adhesion molecules E-selectin, VCAM and ICAM in parallel with a synthetic 3×NF-κB responsive reporter. All three promoters contain several NF-κB responsive elements (RE's) whereas no responsive elements for nuclear receptor are present (see app 1). The Eselectin promoter contains several NF-κB responsive elements comprising those which are optimal for p50/RelA, RelA/RelA and RelA/c-Rel heterodimers [Parry and Mackman, 1994 J. Biol. Chem. 269: 20823-20825; Chen et al., Nature 391, 1998a; Chen et al., Nature Struct. Biol.5: 67-73, 1998b, 1994??; Kunsch et al., 1992, Mol. Cell. Biol. 12: 4412-4421). The 3×NF-κB reporter contains 3 copies of a conserved p50/ReLA response element (Wissink et al, Immunobiol. 198: 50-64, 1997; Wissink et al., Mol Endocrinol. 15(4):543-52, 2001).

All three promoters are clearly induced by cytokines, such as Il-1β and TNFα. In particular, induction of Eselectin by Il-1β was quite powerful (15 fold over unstimulated promoter activity) and similar to induction of the optimized synthetic 3×NF-κB responsive reporter (data not shown). Studies in literature mainly focus on RelA and p50 when studying effects of NF-κB. In many cases this choice may be logical, since inflammatory stimuli result in the induction of the activated RelA-p50 heterodimer, whereas for instance expression and activity of c-Rel is constitutively high in inflammatory cell types such as B and T lymphocytes (Grumont et al.; Cell Growth and Diff. 4: 731-743. 1993; Dobrzanski et al., EMBO J. 13: 4608-4616. 1994). However, the types of responses that are interesting for estrogenic drugs are not the acute inflammatory responses, but rather the more chronic mechanisms involved in the development of atherosclerosis and in the continuous interplay between osteoblasts and osteoclasts during bone homeostasis.

In order to study the effects of different types of NF-κB on the activity of NF-κB responsive promoters, co-transfection experiments were designed in which combinations of promoter-reporter and NF-κB proteins were tested (Table 1). We found that activity of the E-selectin promoter is more potently induced by RelA than by c-Rel. Co-expression of both RetA and c-Rel gave induction of promoter activity that was in between the induced levels by RelA and c-Rel separately, suggesting that RelA (homodimer) is a much more potent inducer than a RelA/c-Rel heterodimer. On the 3×NF-κB responsive reporter, c-Rel containing heterodimers were as potent as RelA containing homo/heterodimers. Effects on both reporters show that in these cases c-Rel by itself (as a homodimer) has negligible transactivating activity. These data imply that c-Rel preferentially acts as a heterodimer with other types of NF-κB proteins in transactivation of the genes studied here.

| | ELAM− | 3*NFkB-reporter |
|---|---|---|
| — | 2969 | 5545 |
| cRel | 4778 | 6248 |
| RelA | 1724355 | 295608 |
| cRel + RelA | 591260 | 300209 |
| cRel + p50 | 381857 | 234211 |
| RelA + p50 | 1692569 | 328423 |

Table 1. Induction of the R-selectin and the 3×NF-κB responsive promoter, as measured by luciferase activity, by different types of NF-κB proteins in U-2 OS osteosarcoma cells. Effects of the indicated NF-κB proteins or combinations thereof on reporter activity were measured by transient co-transfection experiments, as indicated in Materials and Methods. More information on the composition of the reporters is given in appendix 1. The numbers in the table are counts per second reflecting luciferase activity.

Using the promoters of E-selectin, ICAM and VCAM the functional interactions between ER and NF-κB signaling via an NF-κB reporter were studied. All three promoter-reporters can be induced efficiently by Il-1β (Table 2). Following transient transfection, Il-1β induced activity of these NF-κB responsive promoters can be transrepressed in an ERα- and ligand-dependent manner (Table 2). Complete repression was not observed in these experiments; Il-1β induced promoter activity is repressed up to 60% of induced activity. This is similar to the transrepressive effects of glucocorticoids on these promoters that have been determined in parallel (data not shown). Dose-responses for 17β-estradiol show that halfmaximal repression occurs at a concentration in between 10⁻¹¹ and 10⁻¹²M, which is similar to the EC50 value of 17β-estradiol in ERE-transactivation in U-2 OS cells (data not shown). Therefore 17β-estradiol is not selective.

Next was determined whether transrepression by ER is limited to particular NF-κB types, such as c-Rel or RelA, by overexpression of these NF-κB proteins in U-2 OS cells (Table 2). Again, the promoter-reporter constructs for the E-selectin, ICAM and VCAM genes were used in these experiments. Significant activation of these reporters by overexpression of c-Rel was observed. This activation was strongly and reproducibly transrepressed by both ERα and ERβ (Table 2). In contrast, the reporters were strongly induced by RelA but no repression of this activity by ER was observed (Table 2).

| | | | | | 3*NFkB- |
|---|---|---|---|---|---|
| | ELAM+ | ELAM− | ICAM | VCAM | reporter |
| — | 6664 | n.d. | 156630 | 6738 | 106718 |
| IL-1 + ER alpha | 99757 | n.d. | 908172 | 30296 | 1836839 |
| IL-1 + ER alpha + E2 | 58937 | n.d. | 436002 | 20068 | 1383927 |
| — | n.d. | 4281 | n.d. | n.d. | 30606 |
| RelA + ER alpha | n.d. | 951867 | n.d. | n.d. | 682576 |
| RelA + ER alpha + E2 | n.d. | 798454 | n.d. | n.d. | 572861 |
| — | 1038 | 313 | 928 | 183 | 3516 |
| cRel + ER alpha | 6291 | 2468 | 4283 | 1088 | 7133 |
| cRel + ER alpha + E2 | 3256 | 569 | 3514 | 259 | 5140 |
| — | 312 | 1303 | 304 | 179 | n.d. |
| cRel + ER beta | 5839 | 2360 | 1738 | 1738 | n.d. |
| cRel + ER beta + E2 | 3708 | 1495 | 1611 | 882 | n.d. |

Table 2. Estrogen-dependent transrepression of the promoters from E-selectin, VCAM and ICAM and a synthetic NF-κB responsive reporter, measured as the amount of luciferase activity. Transient (co-)transfections of ERα (or ERβ) and reporter were performed in U-2 OS cells. Reporter activity was induced by cellular exposure to Il-1β or by co-transfection of the NF-κB genes c-Rel or RelA. Repression of reporter activity was brought about by treatment of the cells with 17β-estradiol, as indicated in Material and Methods. For detailed information on the composition of the reporters used, see appendix 1. The hyphen (-) in the table indicates the test with cells without transfected cDNA and no additives in medium; n.d. means: not determined.

### Example 2

### Reciprocal Repression of ER and GR Dependent Promoter Activity by NF-κB

From literature (Bodine et al., Endocrinology 140: 2439-2451, 1999), it is known that ER/NF-κB interactions modulate pathways that are induced by NF-κB proteins as well as pathways that are induced by activated ER via conserved estrogen responsive elements (ERE). The previous section showed the repression of NF-κB signaling by ER. Below the repression of ER signaling by NF-κB proteins is described. This was shown using transient transfections of U-2 OS osteoblasts with an ER responsive promoter reporter in combination with ERα or ERβ and either c-Rel or RelA (Table 3). These experiments show that particularly RelA is a strong repressor of ERα-induced ERE promoter activity. Similar results were obtained for ERβ.

Strikingly, there does not seem to be a correlation between the extent of repression of an ERE-versus NF-κB responsive promoter activation. ERα and ERβ are able to repress, albeit to different extent, a c-Rel-induced NF-κB reporter whilst having no effect on a RelA-induced reporter. On the other hand, both c-Rel and RelA can repress an ER-induced ERE reporter. This indicates that a functional cross-talk between ER and NF-κB pathways do occur, but that this may have different consequences for either pathway.

The use of a consensus GRE-responsive reporter system in combination with either RelA or c-Rel showed that co-expression of GR with ReLA resulted in high repressive activity on GRE based reporter systems, while hardly any repression was observed with c-Rel. This is a further illustration of the specificity of the interaction between ER and c-Rel.

| | cRel | | | | RelA | | | |
|---|---|---|---|---|---|---|---|---|
| μg NFkB added: | 0 | 0 | 0.1 | 0.5 | 0 | 0 | 0.1 | 0.5 |
| hormone added/not added: | − | + | + | + | − | + | + | + |
| ER alpha/ERE | 1073 | 75987 | 57516 | 31971 | 1073 | 75987 | 10070 | 3893 |
| ER beta/ERE | 1073 | 10107 | 6243 | 3319 | 1073 | 10107 | 1910 | 1004 |
| GR/GRE | 29501 | 1489156 | 1523058 | 1108396 | 53396 | 2264907 | 19126 | n.d. |

Table 3. Effects of different NF-κB proteins on ER and GR mediated reporter activation, measured as luciferase activity. U-2 OS osteosarcoma cells were transiently (co-)transfected with a combination of ERE/GRE reporter, ERα, ERβ or GR nuclear receptors and different amounts of c-Rel or ReZA encoding cDNA constructs after which reporter activation was determined as outlined in Material and Methods. n.d. means: not determined.

### Example 3

### ER/NF-κB Transrepression and Endogenous Gene Expression

In order to demonstrate the significance of ER/NF-κB interactions in vivo and for drug therapies, functional ER/NF-κB interactions were studied at the levels of endogenous gene expression.

We could not study estrogen effects on endogenous E-selectin and VCAM expression in endothelial cells, since both genes are selective for endothelial cell types whereas endothelial cells with sufficient ERα levels were not available at the time. Therefore, modulation of endogenous expression of E-selectin and VCAM was studied in a functional animal model for atherosclerosis (Table 4). Rabbits, like human, develop atherosclerosis in response to a diet containing high cholesterol. By exposing rabbits to such atherogenic diet for a number of weeks, an increase of both E-selectin and VCAM expression could be seen in all regions of the aorta studied (aorta bow, the abdominal and the thoracic part of the aorta). Importantly, by treatment with 17β-estradiol, these induced levels of Eselectin and VCAM were found to be repressed (Table 4), demonstrating the transrepression of ND-κB signalling by estrogens in vivo.

| Diet | exposure to E2 | | |
|---|---|---|---|
| (weeks) | (hours) | ELAM | VCAM |
| 2 | 0 | 0 | 0.005511 |
| 2 | 4 | 0 | 0.004949 |
| 2 | 12 | 0.0003897 | 0.010327 |
| 4 | 0 | 0.0004012 | 0.005405 |
| 4 | 4 | 0.0001909 | 0.005097 |
| 4 | 12 | 0 | 0.006225 |
| 8 | 0 | 0.0013583 | 0.011064 |
| 8 | 4 | 0.0006813 | 0.007292 |
| 8 | 12 | 0.0001990 | 0.009832 |
| 18 | 0 | 0.0023730 | 0.041416 |
| 18 | 4 | 0.0027879 | 0.034499 |
| 18 | 12 | 0.0008307 | 0.020171 |

Table 4. In vivo measurement of ER-mediated transrepression of expression of the endogenous E-selectin and VCAM genes in the aorta bow. In this experiment, rabbits were exposed for 2, 4, 8 or 18 weeks treatment to atherogenic diet +/-31 17β-estradiol treatment. Estrogen effects were determined after exposure to (3 μg/kg) 17β-estradiol (Org 2317) injected subcutaneously 4 or 12 hours prior to sacrificing the animals. mRNA levels were determined using RNase protection, as described in Materials and Methods.

### Example 4

### A Yeast Two Hybrid Binding Assay for Measuring ER/NF-κB Interactions

An HTS assay can be used to screen for compounds that selectively induce the interaction between ER and c-Rel regardless of the tissue-selective background. For this purpose, an ER/c-Rel protein-protein interaction assay is exemplified using the yeast two-hybrid system, which is suitable for screening purpose.

As full-length ERα shows a strong transcriptional activity, an N-terminally truncated transcriptionally silent ERα variant (delta AB) was used to detect interactions of ERα with c-Rel. Full-length ERβ in fusion to GAL4-DBD does not show any intrinsic activity, so full-length ERβ could be used for the interaction studies.

In this manner, a strong estrogen-dependent interaction of ERα and c-Rel was detected. A similar interaction between ERP and c-Rel was found (Table 5). ERα interaction with c-Rel was dependent on the LBD of ERα, as could be shown using truncated ERα constructs (Table 5). Strikingly, no interaction was found between RelA and ERα nor between RelA and ERβ (Table 5). This is in line with the observation that estrogens did not repress ReLA-induced reporter activity (Table 2], but is in apparent contrast with the finding that overexpression of RelA potently repressed both ERα and ERβ induced ERE-transactivation (Table 3]. The latter may be explained by the presence of a pool of different types of inactive NF-κB proteins (in association with IκB) in the cytoplasm of mammalian cells. Overexpression of one NF-κB type is expected to modify the balance between active and inactive NF-κB proteins and hence might influence homo- and heterodimerization.

The interaction of c-Rel and ERα in the yeast 2-hybrid system was transformed into a high throughput assay. This assay can be used for selection of compounds that are potent inducers of the association of ERα with this NF-κB.

| | conc. E2 (nM): | | | | | | | | | | | |
|---|---|---|---|---|---|---|---|---|---|---|---|---|
| | 0.0003 | 0.001 | 0.003 | 0.01 | 0.03 | 0.1 | 0.3 | 1 | 3 | 10 | 30 | 100 |
| | cRel | | | | | | | | | | | |
| ER alpha delta AB | 969 | 984 | 1110 | 947 | 1105 | 1013 | 1192 | 3846 | 18895 | 34581 | 41133 | 52178 |
| ER alpha | 775 | 913 | 1110 | 958 | 1116 | 1377 | 8241 | 44051 | 88987 | 82537 | 97309 | 87335 |
| ER alpha LBD | 892 | 966 | 977 | 964 | 1002 | 947 | 884 | 1013 | 2618 | 12382 | 35435 | 56769 |
| ER beta | n.d. | n.d. | n.d. | 404 | n.d. | 1818 | n.d. | 2140 | n.d. | 2656 | n.d. | 3216 |
| | RelA | | | | | | | | | | | |
| ER alpha delta AB | n.d. | 100 | n.d. | n.d. | n.d. | 112 | n.d. | n.d. | n.d. | 122 | n.d. | n.d. |
| ER alpha | 1081 | 1320 | 1664 | 1868 | 2414 | 3162 | 4291 | 16681 | 32852 | 46960 | 60692 | 57779 |
| ER alpha LBD | n.d. | 86 | n.d. | n.d. | n.d. | 94 | n.d. | n.d. | n.d. | 208 | n.d. | n.d. |
| ER beta | n.d. | n.d. | n.d. | 130 | n.d. | 144 | n.d. | 158 | n.d. | 182 | n.d. | 156 |
| | no partner | | | | | | | | | | | |
| ER alpha delta AB | 1596 | 1405 | 1635 | 1327 | 1237 | 1034 | 1490 | 1831 | 1982 | 2152 | 2072 | 1963 |
| ER alpha | 882 | 950 | 921 | 887 | 1012 | 933 | 1282 | 8547 | 28913 | 37896 | 46277 | 62709 |
| ER alpha LBD | 737 | 881 | 939 | 872 | 880 | 856 | 723 | 802 | 847 | 819 | 984 | 1115 |
| ER beta | n.d. | n.d. | n.d. | 164 | n.d. | 188 | n.d. | 178 | n.d. | 186 | n.d. | 188 |
| cRel | 1251 | 1095 | 1135 | 1093 | 1202 | 1204 | 1202 | 1145 | 1246 | 1146 | 1411 | 1261 |
| RelA | 1101 | 1212 | 1457 | 1457 | 1699 | 1512 | 1051 | 1225 | 1311 | 1365 | 1641 | 1672 |

Table 5. Hormone dependent interaction between ER and NF-κB proteins, as determined in the yeast two-hybrid system, measured as β-galactosidase activity. In this assay format, ERα (or ERβ) was fused to the DNA binding domain of GAL4 whereas c-Rel (or RelA) was fused to the activation domain of GAL4. Interaction between ER and NF-κB yields a transcriptionally active complex, that induces the LacZ reporter gene which results in quantifiable β-galactosidase activity. n.d. means: not determined.

### Example 5

### Transrepression Assay for Beneficial Effects of ER on Endothelial Cells

To further characterise compounds that will be picked up by the high throughput interaction assay, a functional transrepression assay was required based on the results described in examples 1-3. The original aim when starting the ER/NF-κB transrepression studies with the E-selectin, VCAM and ICAM promoters was to develop an assay with high predictive value for atheroprotective effects of estrogens. Epidemiological studies have shown that premenopausal women run a lower risk to develop atherosclerosis. Several in vitro and in vivo data support the concept that estrogens are protective against cardiovascular disease (reviewed in Skafar et al., J. Clin. Endocr. Metab. 82: 3913-3918. 1997), although clinical studies do not always support these data (Hulley et al., JAMA 280: 605-613, 1998).

One of the mechanisms by which estrogens are thought to be atheroprotective is by inhibition of inflammatory reactions, during development of atherosclerosis, possibly via interference with NF-κB signaling. NF-κB proteins play an important role in expression modulation of inflammatory genes (e.g. cytokines) and cell adhesion molecules (example 1). Expression of cell adhesion molecules can be repressed by estrogens and cytoline induced activity of the ICAM, VCAM and E-selectin promoters can be inhibited by estrogens (example 1).

This example describes an assay, based on the E-selectin or VCAM promoter in combination with ERα in the T24/ECV cell line. Note that for the ER/NF-κB transrepression assay the ECV304 cell line was used, which was believed to be the only endothelial cell line available that was suitable for producing stable ERα expressing clones. At a later stage ATCC reported that the ECV304 cells were no endothelial cells but, instead, were T24 bladder carcinoma cells. In order to enhance the cytokine induction and amount of repression by estrogens, multiple copies of NF-κB responsive regions of the E-Selectin and VCAM promoters were used to construct new reporters named ELAM1a, ELAM2, ELAM-HMG, VCAM1-2, VCAM2, and VCAM3 (for reporter details see appendix 1). Transient transfection of the different E-selectin and VCAM promoter derived reporters showed particularly good results (reproducible high induction by cytokines and more than 50% repression by ERa) for ELAM-HMG and VCAM3 [Table 6].

| | ELAM− | ELAM1a | ELAM2 | ELAM-HMG | VCAM | VCAM1-2 | VCAM2 | VCAM3 |
|---|---|---|---|---|---|---|---|---|
| A | | | | | | | | |
| — | 24458 | 772 | 13824 | 685 | 1560 | 1863 | 4892 | 4357 |
| IL-1 | 160167 | 1818 | 39383 | 12739 | 3099 | 3741 | 19464 | 22767 |
| IL-1 + E2 | 115307 | 929 | 25126 | 6361 | 2205 | 2403 | 12735 | 12238 |
| B | | | | | | | | |
| — | 16767 | 4537 | 3592 | 5848 | 11269 | 3054 | 3011 | 4243 |
| cRel | 149922 | 12926 | 15543 | 218149 | 155411 | 13537 | 11354 | 21983 |
| cRel + E2 | 90035 | 7390 | 6223 | 61571 | 51283 | 9107 | 4783 | 10781 |

Table 6 Selection of the most suitable NF-κB-responsive reporters in ECV304-ERα (A) or COS (B) cells, measured as luciferase activity. ECV304-ERα cells stably express ERα and endogenous c-Rel, whereas COS cells were transiently transfected with ERα, and, where indicated, c-Rel. Both cell types were co-transfected with the indicated E-selectin (ELAM) or VCAM promoter derived reporters. Cells were exposed to IL-1 and 17β-estradiol, as indicated, prior to measuring luciferase activity (cps) as described in Materials and Methods.

These data were confirmed in stably transfected clones expressing ERα in combination with the ELAM-HMG or VCAM3 reporter (Table 7). Unfortunately, the clones proved not suitable for testing a very high number of compounds due to low stability in transrepression measured during prolonged culture of the cells. The latter is in agreement with a recent publication (Quaedackers et al. Endocrinology 142:1156-1166, 2001), showing a decrease in transrepression of NF-κB signaling by ERα in stably transfected cells relative to transiently transfected cells. Therefore, the above approach is not the most optimal choice for the design of a higher throughput transrepression assay.

| | |
|---|---|
| ELAM-HMG | VCAM3 |

| clone | 2F4 | 2B8 | 4B6 | 3A10 |
|---|---|---|---|---|
| — | 124 | 683 | 169 | 333 |
| IL1 | 1566 | 8410 | 2122 | 3907 |
| IL1 + E2 | 978 | 3995 | 1098 | 2068 |

Table 7 Analysis of stably transfected ECV304/T24ERα-ELAM-HMG and ECV304/T24-ERα/VCAM3 clones by measuring luciferase activity in response to either or both Il-1β and 17β-estradiol (E2). ECV304/T24-ERα cells were stably transfected with the ELAM-HMG or VCAM3-reporter and tested as indicated in FIG. 6. Reporter activation was achieved using Il-1β whilst transrepression was induced using 17β-estradiol.

### Example 6

### Transrepression Assay for Beneficial Effects of ER on Osteoblast Cells

Estrogens maintain the balance between bone resorption and formation by inhibiting the production of cytokines by osteoblasts that stimulate osteoclast activity and thus bone resorption. We analysed the relevance of ER NF-κB transrepression on IL-6 production in more depth. When the pre-osteoblast cell line U-2 OS was stimulated with cytokines such as IL-1β, production of endogenous IL-6 protein is increased. However, when U-2OS cells are also exposed to IL-1β and estrogens, the increased production of IL-6 is repressed [Table 8].

| IL-1 + HFN | − | + | + | + | + | + |
|---|---|---|---|---|---|---|
| E2 (nM) | − | − | 0.001 | 0.01 | 0.1 | 1 |
| Il-6 production | 11083 | 127146 | 136039 | 79230 | 50375 | 41070 |

Numbers are luminescence in counts per sec reflecting IL-6 produced by the cells

Table 8 U-2OS cells stably expressing ERα were incubated with IL-1β (125 pg/ml)+IFNγ (10 ng/ml) and variable amounts of 17β-estradiol (0, 10⁻¹²M to 10⁻⁹M). Secretion of endogenously produced IL-6 was measured by sandwich ELISA as outlined in Material and Methods.

To determine the role of functional ERα/c-Rel interactions in repression of IL-6 production by estrogens, we analysed the effect of various combinations of ERα and NF-κB proteins on IL-6 production using a reporter construct containing the promoter of the IL-6 gene. This analysis again confirms the specificity of ERa for the NF-κB protein. Reporter induction by c-Rel could strongly be repressed by estrogen-stimulated ER, whereas no effect was noted on RelA-induced reporter activity [Table 8]. Also in line with literature [Ray and Prefontaine Proc. Natl. Acad. Sci. 91: 752-756, 1994; Stein and Yang, Mol. Cell. Biol. 15: 4971-9, 1995], we found that estrogens display transrepression effects on c/EBPβ [Table 9]. In particular, c-Rel and c/EBPβ synergistically induce the IL-6 promoter-reporter, and this can strongly be repressed by estrogens. The finding that similar synergistic effects between RelA and c/EBPβ could not be repressed by estrogens, again stresses the importance of c-Rel/ERa interaction in repression of NF-κB induced gene expression.

| | — | E2 |
|---|---|---|
| — | 1488 | 1310 |
| p50 | 1824 | 1447 |
| cRel | 3872 | 1807 |
| RelA | 14803 | 19401 |
| C/EBPβ | 4527 | 2585 |
| cRel + p50 | 2485 | 1814 |
| RelA + p50 | 7060 | 8232 |
| c/EBPβ + p50 | 2950 | 1724 |
| cRel + RelA | 18223 | 23381 |
| cRel + c/EBPβ | 31332 | 6459 |
| RelA + c/EBPβ | 91717 | 92837 |

Table 9 Analysis of the effect of ER/NF-κB interactions on activation of the IL-6promoter, measured as luciferase activity. A reporter construct containing the IL-6 promoter region was transiently transfected in U-2 OS cells, together with ERα and various NF-κB proteins (c-Rel, RelA, p50) and c/EBPβ as indicated and incubated with 17β-estradiol or control medium.

### Example 7

### Selectivity of Compounds for Inducing ER/cRel Interactions Over ERE-Based Transcriptional Activity

In example 4 a yeast two hybrid method has been described for the use of selection of compounds that are potent inducers of interaction between estrogen receptor and c-Rel. Table 10 shows typical results for 4 compounds in this assay format. In order to test the selectivity of these compounds in ER/c-Rel interactions over inducing direct transcriptional activity of ER, the compounds were tested in a dilution series on repression of cytokine induced ELAM-HMG promoter reporter activity in T24/ECV cells (see example 5 for more details on assay format). In parallel, dilution series of the compounds were tested in ERE transactivation in CHO or U-2 OS cells.

For measuring ERE transactivation in U-2 OS, the cells were transiently transfected with ERα and ERE-based promoter reporter construct pRo-luc. The latter construct contains part of the rat oxytocin promoter that is representative for ER transcriptional activity due to a conserved estrogen responsive element in the promoter (see appendix 2). The used part of the oxytocin promoter does not contain NF-κB responsive elements. For ERE transactivation in CHO cells, a CHO cell clone that is stably transfected with ERα and pRoLuc was used.

Based on the dose-response curves produced for the above assays, EC50 values were determined for all 3 compounds and for 17β-estradiol. EC50 values are the concentration at which half-maximum effect of the compound is observed. In the used assays this may either be half-maximum repression (in the case of ELAM-HMG reporter) or half-maximum induction (in the case of the interaction between ERα and c-Rel in yeast two hybrid or in the case of ERE-reporter activity).

Results in table 10 show that Org 38633, Org 38635 and Org10217 display selectivity for ERα/cRel transrepression (ELAM-HMG reporter in T24/ECV cells) over transcriptional activity (ERE-reporter in CHO cells). The natural hormone for ERα (17β-estradiol) does not show this selectivity and may even be less potent in ER/c-Rel transrepression than in ER/ERE transactivation.

| compound | ER/cRel Y2H | ER/cRel functional | | ER/ERE functional |
|---|---|---|---|---|
| Org 38633 | 100 nM | 0.3/0.3 | nM | 4.3¹⁾/5²⁾/0.3²⁾ |
| Org 38635 | 500 nM | 0.7/0.4 | nM | 3.4¹⁾/2.3²⁾ |
| Org 10217 | 5 nM | 0.03/0.04/0.02 | nM | 0.5¹⁾ |
| 17β-estradiol | 5 nM | 0.03/0.07/0.05 | nM | 0.01²⁾/0.02²⁾ |

table 10. Analysis of EC50 values (expressed in nLM) of three compounds (Org 38633, Org 38635 and Org 10217) in comparison to 17β-estradiol in induction of ERα/c-Rel interactions versus ER transcriptional activity. First column depicts the internally used organon codes of these compounds. EC50 values of the compounds in the ER/cRel yeast 2-hybrid assay are shown in the second column. The third column gives the EC50 values in the functional ERα/c-Rel interaction assay using the ELAM-HMG promoter reporter in the presence of ERα in T24/ECV cell line (see example 5for details). In the last column the EC50 values in ERa transcriptional activity is shown. The latter is either measured in: 1) U-2 OS cells that have been transiently transfected with ERα and an ERE-TATA-Luc reporter (indicated with ¹⁾) or 2) in CHO cells that are stably transfected with ERα and the ERE-RO-Luc reporter (indicated with ²⁾).

5. Materials and Methods

Cell Culture

Human osteosarcoma U-2 OS (HTB-96), monkey kidney COS-1 (CRL-1650) and human ECV304 (CRL-1998) cells have been obtained from ATCC (American Type Culture collection). ECV304 cells were sold by ATCC as human endothelial cells, however at a later stage ATCC informed us that this cell line was in fact the human bladder epithelial carcinoma cell line T24. Therefore, these cells are called T24/ECV in this report. ECV304/T24(-ERα), U-2 OS and COS-7 cells were grown in a 1:1 mixture of phenol red free Dulbecco's modified medium (DMEM) and Ham's F12 medium (=M505), supplemented with 10% fetal calf serum (FCS). Experiments were performed in M505+10% FCTS (fetal calf serum treated with dextran-coated charcoal to deplete serum from steroid activity).

Constructs Used

pKCRE-ERα, pKCRE-ERβ and pNGV1-GR contain the human wild-type estrogen receptor α (ERα), ERβ or GR respectively under control of the SV40 promoter. The pCMV4-c-Rel expression vector was produced by cloning of the coding region of c-Rel into the pCMV4 vector (Andersson et al., 1989). pGL3-ELAM, pGL3-VCAM and pGL3-ICAM have been constructed based on PCR using the sequences as outlined in appendix 1. The luciferase reporter plasmid 3*NF-κB-TK was constructed by inserting three repeats of the NF-κB binding site from the ICAM promoter (van de Stolpe et al.; J. Biol Chem. 269: 6185-6192. 1994) in front of the herpes simplex virus thymidine kinase (HSV-TK) promoter. The TK promoter was cloned into the Bgl II-site of pGL3-basic (Promega). The triple repeat of NF-κB binding sites was constructed by annealing the oligonucleotide primers 5′-catacggtaagcttggggtcatcg ccctgccaccgccgcccgattgctttagcttggaaattccgga-3′ and 5′-gtatgccaaagcttctccggaatttccaagctccg gaatttccaagctccggaatttccaagctaaa-3′ followed by PRC amplification and subcloning in the pCR™2.1 vector. The insert was excised using HindIII, blunted with Klenow DNA polymerase and ligated into the SmaI site of pGL3-tk-luc. 4xERE-TATA Luc (plasmid nr. 460) contains four repeats of an estrogen responsive element followed by a TATA box and a luciferase gene. The pSV-β-Galactosidase (pSVβ) control vector (Promega) served as a positive control vector for monitoring transfection efficiencies of mammalian cells. The SV40 early promoter and enhancer drive transcription of the LacZ gene which encodes the β-Galactosidase enzyme. The yeast 2-hybrid constructs pGAD424-ERα, pGAD424-ERαdAB and pGAD424-ERβ were constructed by cloning respectively the human ERα, the ERα without the AB domain and the human ERβ cDNA in pGAD424. pGAD424-ERα LBD was constructed by cloning the LBD of ERα, obtained by PCR, in the EcoRI and SalI sites of pGAD424 vector. pGBT9-c-Rel was constructed by cloning the c-Rel fragment, obtained by PCR on pCMV4-c-Rel in the EcoRI and BamHI sites of pGBT9. The p65/RelA cDNA was obtained by PCR on the pCMV4-p65. This fragment was cloned in the pGEX4T1, which was used for subcloning p65/RelA cDNA in the blunted XmaI site of pGAD424.

The six NF-κB responsive reporters pHGL3-ELAM 1a, pHGL3-ELAM 2, pHGL3-ELAM HMG, pHGL3-VCAM 1-2, pHGL3-VCAM 2 and pHGL3-VCAM 3 (plasmid nr. 1921-1926 respectively) were constructed by ligating annealed primers in the Kpn I and Sac I sites of pHGL3-TATA. The primers were based on the NF-κB-RE sequences in the ELAM and the VCAM promoter (see Appendix 1). Several copies of the NF-κB-RE's were cloned in order the gain a higher absolute luciferase signal. The ELAM HMG construct contains a double repeat, while the other constructs contain three NF-κB -RE's. For primer and reporter sequences, see appendix 1.

Transfection

Transient Transfection

For transient transfections, cells were seeded in 6-well plates (2.10⁵ cells/well). After two or three days the cells were transfected with reporter, expression vectors and β-galactosidase control vector. Two different experimental settings were used. For testing transrepression of IL-1β induced promoter activity, cells were transfected with 1 μg reporter construct, 1 μg expression vector (containing either ERα, ERβ or GR), and 0.25 μg of β-galactosidase control plasmid. For testing transrepression in combination with c-Rel or ReLA overexpression, cells were transfected with 1 μg reporter construct, 1 μg expression vector (containing either ERα, ERβ or GR) and 0.1 or 1.0 μg expression vector containing c-Rel or RelA and 0.25 μg of β-galactosidase control vector. Equivalent amounts of DNA were obtained by adding the appropriate expression vector that lacks an insert. Transient transfection was performed using lipofectin reagent (Life Technologies). Transient transfections with lipofectin were performed according to the suggestions of the manufacturer, with some minor changes. Per μg of DNA to be transfected, 5 μl of lipofectin was used and cells were incubated with the transfection mixture during 5 hours, after which the transfection mix was aspirated and replaced by M505+0.5 μg/ml insuline+5 μg/ml transferine+test compound (e.g. different combinations of IL-1β and 17β-estradiol). The transfected cells were incubated overnight with test compounds prior to cell lysis. Measurement of luciferase and P-galactosidase activity were performed using respectively the luciferase assay system (Promega) and Galacto-light plus (Tropix) according to the manufacturers' protocols and measured on the Victor 1420 multilabel counter, Wallac. Detection of β-galactosidase activity was used as a control for transfection efficiency.

Stable Transfection

Cells were seeded in a 60-mm dish in a concentration of 1.10⁶ cells/dish and a total volume of 10 ml. After two days of growth the cells were transfected with the appropriate plasmid and if needed a selection plasmid such as PAG60 (in a 10:1 ratio) was added. The plasmids were added to 200 μl OptiMem. To 25 μl of Lipofectin (Gibco) 175 μl of OptiMem was added and incubated for 30 minutes at room temperature. The DNA/OptiMem solution was added to the Lipofectin solution and incubated for 15 minutes room temperature. Then 3.6 ml OptiMem was added. The cells were washed twice with 5 ml M505 and the transfection solution was added to the cells. After five hours of transfection the medium was replaced by M505+10% FCS. The cells were incubated for two days, trypsinised and divided over two 96 well plates in M505+10% FCS +antibiotic (Neomycin for the pKCRE-ER/PAG 60 cotransfection or Hygromycin for the pHGL3-NF-κB-RE reporter plasmids). Medium was refreshed two times per week. Antibiotic resistant colonies were scored after about two weeks. The T24-ECV cells transfected with pKCRE-ER were checked for the presence of ERα by transient transfection with 3*ERE-TATA-Luc and incubation with 17β-estradiol. The T24-ECV and the T24-ECV-ERα cells transfected with pHGL3-ELAM HMG or pHGL3-VCAM3 cells were checked for the presence of the NF-κB-RE reporter plasmid by incubation with IL-1β and with IL-1β+E2 to score the clones with the highest percentage transrepression.

RNase Protection Assay

Animal Studies and Cell Culture

The experimental protocol for the treatment of rabbits has been previously described (Santegoets et al, 1997). In short, New Zealand White rabbits (2 rabbits per experimental group) were ovariectomized and afterwards received cholesterol enriched diet for 2, 4, 8 or 18 weeks. 4 or 12 hours prior to sacrificing the animals, the animals were injected with 3 μg/kg 17β-estradiol (Org 2317) subcutaneously or received placebo treatment.

Cell culture for the RNase protection studies was performed in 175 cm² flasks. T24-ECV, T24-ECV-ERα-ELAM HMG 2B8 and T24-ECV-ERα-VCAM 3 3A10 cells were grown in M505 supplemented with 10% FCTS for three days, after which cells were stimulated for six hours with either or both 100 U/ml IL1β and 1.10⁻⁸ M 17β-estradiol diluted in M505 (supplemented with 0.1% BSA).

RNA Isolation

Isolation of total RNA from tissue samples or T24/ECV-ERα cells was performed using RNAzol B (Campro Scientific), according to the recommendations of the manufacturer. After incubation with compounds the T24/ECV-ERα cells were trypsinised, resuspended in M505+10% FCS and counted. Next the cells were washed with cold PBS and the RNA was isolated from the cellpellet using RNAzolB. The RNA was quantified by A260 measurement and purity analyzed by determination of the A260/A280 ratio. RNA was stored in 70% ethanol and 0.1M NaAc at −20° C.

RNase Protection Probes

RNase Protection probes used for measuring the expression levels of different sets of cytokines, included the commercial probe sets hCK-3, 4 and 5 from RiboQuant (Pharmingen). For RNase protection on rabbit aorta, probes for VCAM and E-selectin were prepared by RT-PCR. First stand cDNA synthesis was done on 2.5 μg rabbit aortabow total RNA using Superscript II reverse transcriptase (Life Technologies) and random hexanucleotides, according to the recommendations of the manufacturer. PCR was carried out using taq polymerase (Pharmacia) according to the recommendation of the manufacturer. Primers that were used were GATGAGGCCAGTGCTTATTGTCAGC and GCTCACACTTGAGTCCACTGAAGCC for E-selectin and CTCTTACCTGTGCACAGCAAC and CAGTAAATGGTTTCTCTTGAAC for VCAM. Fragments of the appropriate length were subcloned in PCR-2.1 and the sequence identity reconfirmed by sequencing. Probe templates were produced by PCR using a combination of a T7 and an insert-specific primer and purification of the PCR product with the Qiagen gel extraction kit. Primers that were used were TAATACGACTCACTATAGGG for T7 and the above mentioned primers for E-selectin and VCAM respectively. The length of the unprotected/protected RNase protection probes was as follows: E-selectin 494/426 bp, VCAM 717/649 bp, GAPDH 186/118 bp respectively.

Probe Labeling

Antisense RNA probes were synthesized from the templates, which were prepared as described above, using the In Vitro Transcription kit (Pharmingen), T7 RNA polymerase and the incorporation [α-³²P]UTP. Radioactive nucleotides were obtained from NEN™ Life Science Products Inc. Transcription was carried out in a 10 μl reaction containing 20 U RNasin, GTP, ATP and CTP: 137.5 μM each, 3.05 μM UTP, 1.67 μM [α-³²P]UTP (3000 Ci/mmol), 10 μM DTT, 2 μl 5× transcription buffer, 10, 15 and 5 ng ds DNA template for E-selectin, VCAM and GAPDH respectively and 10 U T7 RNA polymerase, at 37° C. for 1 hr. Termination of the transcription was done by adding 1 U DNase, followed by a phenol-chloroform:isoamyl alcohol extraction and an ethanol precipitation. Probe activity was measured in a TRI-CARB liquid scintillation analyzer (Packard).

RNase Protection Analysis

RNase protection was carried out using the RPA kit (Pharmingen). 10 μg of total RNA was hybridized with ca 3×10⁵ cpm of each probe, for 16 h at 56° C. Samples were then subjected to RNase digestion at 30° C. for 45 min. Protected fragments were analysed on a 6% polyacrylamide gel, using the Genemyx LR electrophoresis system (Genomyx). Electrophoresis was carried out for 2 hrs at 2500 V and 125 W at a constant temperature of 50° C. RNase protection results were quantified with the aid of a PhosphorImager Storm 840 and accompanying ImageQuant software (Molecular Dynamics). After quantification, correction of the exact amount of RNA used was performed by dividing the signals of each respective protected band by the GAPDH signal.

As a control for linear correlation between the amount of RNA present and the amount of probe used for hybridization, a control experiment was performed in parallel to measuring vcam and E-selectin expression in the rabbit tissue samples, measuring the hybridisation signal when increasing amounts of liver RNA were used. After the above described correction for GAPDH expression, this resulted indeed in equal amounts of mRNA encoding for E-selectin and VCAM (results not shown).

Yeast Two-Hybrid

Transformation of the yeast strain SFY526 with the pGAD424, containing the different ER constructs, and the pGBT9, containing the c-Rel or RelA construct, was carried out using the Lithium Acetate method. Five individual clones were picked and tested with a concentration series of 17β-estradiol in an P-Galactosidase assay using the Gal Screen™ Reporter Gene Assay System (Tropix, cat. no. GSY 1000). Selection of the best clone was based on 17β-estradiol responsiveness.

Detection of IL-6 Protein

For studying repression of human IL-6 secreted by U-2 OS cells that stably overexpress ERα (U-2 OS-ERα cells) a standard sandwich ELISA was used (ELISA kit of R&D systems #DY206). Cells were plated in 24 or 96 wells plates in M505+dextran-coated charcoal stripped serum (i.e. 10% BCTS) and allowed to grow to 90% confluency. Cells were treated overnight with cytokines, such as different combinations of Il-1β TNFα and IFNγ, in the absence or presence of 17β-estradiol or other ER modulating compounds.

ELISA was performed according to the protocol of the manufacturer and as briefly described below. After overnight incubation of the cells with compounds in the appropriate concentration, the supernatants were transferred to 96 wells plates that had been coated overnight with anti-IL-6 capture antibody. Captured IL-6 was subsequently detected using a biotinylated anti-IL-6 detection antibody and Streptavidin-AP. Il-6 concentrations per well were subsequently quantified using the chemoluminescent substrate CSPD (Elisa-Light™, #EL100CY, Tropix), after which luminescence was determined in a Victor multilabel counter (Wallac).

Appendix 1

Sequence of the cloned parts of respectively the E-selectin, VCAM and IL-6 promoters. The ATF-2-site is marked as dotted underlined, consensus NF-κB—sites as single underlined, c/EBPβ site in double underlined and transcnption initiation sites are marked in bold. The coordinates are relative to the transcription initiation sites.

| ELAM-1 | |
|---|---|
| 160 | GAGTTTC [symbol] TTGTA ATTTTAAGCA TCGTGGATAT |
| | TCCCGGGAAA GTTTTTGGAT CCATTGGGG ATTTCCTCTT |
| 80 | TACTGGATGT GGACAATATC CTCCTATTAT TCACAGGAAG |
| | CAATCCCTCC TATAAAAGGG CCTCAGCCGA AGTAGTGTTC |
| 0 | AGCTGTTCTT GGCTGACTTC |
| ICAM-1 | |
| 617 | GTTAGCGGTC GCCGGGAGGT GCCTGGCTCT GCTCTGGCCG |
| | CTTCTCGAGA AATGCCCGTG TCAGCTAGGT GTGGACGTGA |
| 537 | CCTAGGGGGA GGGGCATCCC TCAGTGGAGG GAGCCCGGGG |
| | AGGATTCCTG GGCCCCCACC CAGGCAGGGG GCTCATCCAC |
| 457 | TCGATTAAAG AGGCCTGCGT AAGCTGGAGA GGGAGGACTT |
| | GAGTTCGGAC CCCCTCGCAG CCTGGAGTCT CAGTTTACCG |
| 377 | CTTTGTGAAA TGGACACAAT AACAGTCTCC ACTCTCCGGG |
| | GAAGTTGGCA GTATTTAAAA GTACTTAATA AACGCCTTAG |
| 297 | CGCGGTGTAG ACCGTGATTC AAGCTTAGCC TGGCCGGGAA |
| | ACGGGAGGCG TGGAGGCCGG GAGCAGCCCC CGGGGTCATC |
| 217 | GCCCTGCCAC CGCCGCCCGA TTGCTTTAGC TTGGAAATTC |
| | CGGAGCTGAA GCGGCCAGCG AGGGAGGATG ACCCTCTCGG |
| 137 | CCCGGGCACC CTGTCAGTCC GGAAATAACT GCAGCATTTG |
| | TTCCGGAGGG GAAGGCGCGA GGTTTCCGGG AAAGCAGCAC |
| 57 | CGCCCCTTGG CCCCCAGGTG GCTAGCGCTA TAAAGGATCA |
| | CGCGCCCCAG TCGACGCTGA GCTCCTCTGC TAC |
| VCAM-1 | |
| 102 | AAACTTTTTT CCCTGGCTCT GCCCTGGGTT TCCCCTTGAA |
| | GGGATTTCCC TCCGCCTCTG CAACAAGACC CTTTATAAAG |
| 22 | CACAGACTTT CTATTTCACT CCGCGGTATC TGCATCGGGC |
| | CTCACTGGCT |
| IL-6 | |
| 288 | GCTAGCCTCA ATGACGACCT AAGCTGCACT TTTCCCCCTA |
| | GGGTGTCTT |
| 238 | GCGATGCTAA AGGACGTCAC ATTGCACAAT CTTAATAAGG |
| | TTTCCAATCA |
| 188 | GCCCCACCCG CTCTGGCCCC ACCCTCACCC TCCAACAAAG |
| | ATTTATCAAA |
| 138 | TGTGGGATTT TCCCATGAGT CTCAATATTA GAGTCTCAAC |
| | CCCCAATAAA |
| 88 | TATAGGACTG GAGATGTCTG AGGCTCATTC TGCCCTCGAG |

Sequences of the primers which were used for construction of the pHGL3-ELAM 1a, pHGL3-ELAM 2, pHGL3-ELAM HMG, pHGL3-VCAM 1-2, pHGL3-VCAM 2 and pHGL3-VCAM 3reporters.

PRIMERS

| | |
|---|---|
| TATA-box | 5′-ATCCTCGAGCAATCCCTCCGGGTATATAATAAGCTT |
| | ATC-3′ |
| | 5′-GATAAGCTTATTATATACCCGGAGGGATTGCTCGAG |
| | GAT-3′ |
| ELAM 1a | 5′-TCGGTACCTGGATATTCCCGGGTCGTGGATATTCCCGGG |
| | TCGTGGATATTCCCGAGCTCATC-3′ |
| | 5′-GATGAGCTCGGGAATATCCACGACCCGGGAATATCCACG |
| | ACCCGGGAATATCCAGGTACCGAT-3′ |
| ELAM 2 | 5′-ATCGGTACCGGGGATTTCCTCTTAATTGGGGATTTCCTC |
| | TTAATTGGGGATTTCCTCGAGCTCATC-3′ |
| | 5′-GATGAGCTCGAGGAAATCCCCAATTAAGAGGAAATCCCC |
| | AATTAAGAGGAAATCCCCGGTACCGAT-3′ |
| ELAM | 5′-ATCGGTACCTGTAATTTTAAGCATCGTGGATATTCCCGG |
| HMG | GAAAGTTTTTTGTAATTTTAAGCATCGTGGATATTCCCGGGA |
| | AAGTTTTGAGCTCATC-3′ |
| | 5′-GATGAGCTCAAAACTTTCCCGGGAATATCCACGATGCTT |
| | AAAATTACAAAAAACTTTCCCGGGAATATCCACGATGCTTAA |
| | AATTACAGGTACCGAT-3′ |
| VCAM | 5′-ATCGGTACCTGGCTCTGCCCTGGGTTTCCCCTTGCCCTG |
| 1-2 | GCTCTGCCCTGGGTTTCCCCGAGCTCATC-3′ |
| | 5′-GATGAGCTCGGGGAAACCCAGGGCAGAGCCAGGGCAAG |
| | GGGAAACCCAGGGCAGAGCCAGGTACCGAT-3′ |
| VCAM 2 | 5′-ATCGGTACCGGGTTTCCCCTTGACTGGGTTTCCCCTTGA |
| | CTGGGTTTCCCCGAGCTCATC-3′ |
| | 5′-GATGAGCTCGGGGAAACCCAGTCAAGGGGAAACCCAGTC |
| | AAGGGGAAACCCGGTACCGAT |
| VCAM 3 | 5′-ATCGGTACCAGGGATTTC₃₄CTCCTGAAGGGATTTCCCT |
| | CCTGAAGGGATTTCCCGAGCTCATC-3′ |
| | 5′- GATGAGCTCGGGAAATCCCTTCAGGAGGGAAATCCCTT |
| | CAGGAGGGAAATCCCTGGTACCGAT-3′ |

Appendix 2
ERE Consensus: GGTCA NNN TGACC

Sequence of the cloned parts of the rat oxytocin promoter as used for measuring transcriptional activity of estrogen receptors. Based on the published ERE palindromic consensus site (GGTCA NNN TGACC), the promoter contains two relatively well conserved ERE's (indicated as bold). The most upstream ERE contains two mismatches, the second one only one. In addition, several half sites are present (indicated as underlined).

1 TACCCGGGAT CCACCGTCGG TGATGGTTTC TCCAGCCCAG ACCGACCTTT
TTATGCCTTG TCCACTGCCA TGGTGGGGCC CAGTCTAAGA GGGTGACTGC
ATGACTGGTC ACAGCCAGGT CTCTTGGGTC AAACTGTTCC ACACTGTTTA
GAAGCAGGCC CTTCATTTGC AGGGTCTGGG CTGGGGTCAA
GGTCACCGCC TCAGCTAATG ACCTGAGCTC AAAAGGGACA CAGCCTAGAA
GGGGAGGCCT AAGCTACAAG AGGATAAAGA GACTTGGAGG
GGGTAGAGGT GCAGCCTAGC CAAGAGCTGT TTTTTCATAG AAATCCAATA
CCTCAGAATG AGGTTGGATA GCGCAAGTGG GTGAGGAAGC CCTTACGTGG
ATCTAAAGCT GACCTGCAGG CATGCAAGC

1. A method to measure a direct interaction between an estrogen receptor and a transactivation protein in an in vitro system, comprising:
providing for a detection parameter proportionally related to the degree of the interaction, wherein the transactivation protein is c-Rel.

2. The method according to claim 1, wherein the method is a yeast two-hybrid assay.

3. The method according to claim 1, wherein the method is for selection, out of a number of compounds, of a compound for therapeutic efficacy in osteoporosis by carrying out the method repeatedly, optionally in parallel, with the presence of each of the number of compounds in the system and selecting a compound which enhances the interaction.

4. The method according to claim 3, wherein the method further comprises testing the compound which enhances the interaction in an animal model for osteoporosis.

5. A method to influence the direct interaction between an estrogen receptor (ER) and c-Rel in a system, comprising:
adding a compound to the system, which compound influences the direct interaction between an estrogen receptor (ER) and c-Rel.

6. A method to influence a direct interaction between an estrogen receptor and a transactivation protein in an in vitro system, comprising:
adding a compound to the system, which compound influences the direct interaction between the estrogen receptor and the transactivation protein, characterised in that the transactivation protein is c-Rel.

7. The method according to claim 5, wherein the compound influences the interaction selectively in comparison to an influence of the compound on ER transcriptional activity.

8. The method according to claim 6, wherein the compound influences the interaction selectively in comparison to an influence of the compound on ER transcriptional activity.

9. (canceled)

10. (canceled)

11. (canceled)

12. The method according to claim 1, wherein the estrogen receptor is ERα or ERβ.

13. The method according to claim 12, wherein the estrogen receptor is the ERα.


SEQUENCE LISTING (39 sequences)

SEQ ID NO: 1 - DNA, 67 bp, Artificial Sequence
  Primer, page 25 line 29-30
catacggtaagcttggggtcatcgccctgccaccgccgcccgattgctttagcttggaaattccgga

SEQ ID NO: 2 - DNA, 67 bp, Artificial Sequence
  Primer, page 25 line 31
gtatgccaaagcttctccggaatttccaagctccggaatttccaagctccggaatttccaagctaaa

SEQ ID NO: 3 - DNA, 25 bp, Artificial Sequence
  Primer, first on page 28 line 37
gatgaggccagtgcttattgtcagc

SEQ ID NO: 4 - DNA, 25 bp, Artificial Sequence
  Primer, second on page 28 line 37
gctcacacttgagtccactgaagcc

SEQ ID NO: 5 - DNA, 21 bp, Artificial Sequence
  Primer, page 29, line 1
ctcttacctgtgcacagcaac

SEQ ID NO: 6 - DNA, 22 bp, Artificial Sequence
  Primer, Page 29 line 2
cagtaaatggtttctcttgaac

SEQ ID NO: 7 - DNA, 20 bp, Artificial Sequence
  Primer, page 29, line 7
taatacgactcactataggg

SEQ ID NO: 8 - DNA, 179 bp, Artificial Sequence
  ELAM-1 sequence of cloned part, page 31, lines 11-15
gagtttctgacatcattgtaattttaagcatcgtggatattcccgggaaagtttttggatccattggggatttcctctttactggatgtggacaatatcctcctattattcacaggaagcaatccctcctataaaagggcctcagccgaagtagtgttcagctgttcttggctgacttc

SEQ ID NO: 9 - DNA, 20 bp, Artificial Sequence
  Underlined consensus sequence page 31 line 11-12
ggatattcccgggaaagttt

SEQ ID NO: 10 - DNA, 12 bp, Artificial Sequence
  Underlined consensus sequence page 31 line 12
ggggatttcctc

SEQ ID NO: 11 - DNA, 633 bp, Artificial Sequence
  ICAM-1 sequence of cloned part, page 31 lines 20-35
gttagcggtcgccgggaggtgcctggctctgctctggccgcttctcgagaaatgcccgtgtcagctaggtgtggacgtgacctagggggaggggcatccctcagtggagggagcccggggaggattcctgggcccccacccaggcagggggctcatccactcgattaaagaggcctgcgtaagctggagagggaggacttgagttcggaccccctcgcagcctggagtctcagtttaccgctttgtgaaatggacacaataacagtctccactctccggggaagttggcagtatttaaaagtacttaataaacgccttagcgcggtgtagaccgtgattcaagcttagcctggccgggaaacgggaggcgtggaggccgggagcagcccccggggtcatcgccctgccaccgccgcccgattgctttagcttggaaattccggagctgaagcggccagcgagggaggatgaccctctcggcccgggcaccctgtcagtccggaaataactgcagcatttgttccggaggggaaggcgcgaggtttccgggaaagcagcaccgccccttggcccccaggtggctagcgctataaaggatcacgcgccccagtcgacgctgagctcctctgctac

SEQ ID NO: 12 - DNA, 10 bp, Artificial Sequence
  Underlined consensus sequence page 31 line 20
gggaggtgcc

SEQ ID NO: 13 - DNA, 10 bp, Artificial Sequence
  Underlined consensus sequence page 31, line 22
ggggcatccc

SEQ ID NO: 14 - DNA, 10 bp, Artificial Sequence
  Underlined consensus sequence page 31 line 29
ggagcagccc

SEQ ID NO: 15 - DNA, 10 bp, Artificial Sequence
  Underlined consensus sequence on page 31 line 30-31
tggaaattcc

SEQ ID NO: 16 - DNA, 130 bp, Artificial Sequence
  VCAM-1, Sequence of cloned part on page 32 lines 4-7
aaacttttttccctggctctgccctgggtttccccttgaagggatttccctccgcctctgcaacaagaccctttataaagcacagactttctatttcactccgcggtatctgcatcgggcctcactggct

SEQ ID NO: 17 - DNA, 10 bp, Artificial Sequence
  Underlined consensus sequence page 32, line 4
gggtttcccc

SEQ ID NO: 18 - DNA, 11 bp, Artificial Sequence
  Underlined consensus sequence onpage 32 lines 4-5
agggatttccc

SEQ ID NO: 19 - DNA, 240 bp, Artificial Sequence
  IL-6, Sequence of cloned part on page 32, lines 14-22
gctagcctcaatgacgacctaagctgcacttttccccctagttgtgtcttgcgatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctcattctgccctcgag

SEQ ID NO: 20 - DNA, 14 bp, Artificial Sequence
  Double underlined c/EBPbeta site on page 32 line 16
acattgcacaatct

SEQ ID NO: 21 - DNA, 10 bp, Artificial Sequence
  Underlined consensus sequence on page 32 line 20
gggattttcc

SEQ ID NO: 22 - DNA, 39 bp, Artificial Sequence
  Primer TATA-box on page 33 first sequence
atcctcgagcaatccctccgggtatataataagcttatc

SEQ ID NO: 23 - DNA, 39 bp, Artificial Sequence
  Primer TATA-box page 33 second sequence
gataagcttattatatacccggagggattgctcgaggat

SEQ ID NO: 24 - DNA, 62 bp, Artificial Sequence
  Primer ELAM 1a on page 33, third sequence
tcggtacctggatattcccgggtcgtggatattcccgggtcgtggatattcccgagctcatc

SEQ ID NO: 25 - DNA, 63 bp, Artificial Sequence
  Primer ELAM 1a on page 33 fourth sequence
gatgagctcgggaatatccacgacccgggaatatccacgacccgggaatatccaggtaccgat

SEQ ID NO: 26 - DNA, 66 bp, Artificial Sequence
  Primer ELAM 2 on page 33 fifth sequence
atcggtaccggggatttcctcttaattggggatttcctcttaattggggatttcctcgagctcatc

SEQ ID NO: 27 - DNA, 66 bp, Artificial Sequence
  Primer ELAM 2 on page 33 sixth sequence
gatgagctcgaggaaatccccaattaagaggaaatccccaattaagaggaaatccccggtaccgat

SEQ ID NO: 28 - DNA, 97 bp, Artificial Sequence
  Primer ELAM HMG on page 33 seventh sequence
atcggtacctgtaattttaagcatcgtggatattcccgggaaagttttttgtaattttaagcatcgtggatattcccgggaaagttttgagctcatc

SEQ ID NO: 29 - DNA, 97 bp, Artificial Sequence
  Primer  ELAM HMG on page 33 eightth sequence
gatgagctcaaaactttcccgggaatatccacgatgcttaaaattacaaaaaactttcccgggaatatccacgatgcttaaaattacaggtaccgat

SEQ ID NO: 30 - DNA, 68 bp, Artificial Sequence
  Primer VCAM 1-2 on page 33 ninth sequence
atcggtacctggctctgccctgggtttccccttgccctggctctgccctgggtttccccgagctcatc

SEQ ID NO: 31 - DNA, 68 bp, Artificial Sequence
  Primer VCAM 1-2 on page 33 tenth sequence
gatgagctcggggaaacccagggcagagccagggcaaggggaaacccagggcagagccaggtaccgat

SEQ ID NO: 32 - DNA, 60 bp, Artificial Sequence
  Primer VCAM 2 on page 33 eleventh sequence
atcggtaccgggtttccccttgactgggtttccccttgactgggtttccccgagctcatc

SEQ ID NO: 33 - DNA, 60 bp, Artificial Sequence
  Primer VCAM 2 on page 33  twelfth sequence
gatgagctcggggaaacccagtcaaggggaaacccagtcaaggggaaacccggtaccgat

SEQ ID NO: 34 - DNA, 63 bp, Artificial Sequence
  Primer VCAM 3 on page 34 first sequence
atcggtaccagggatttccctcctgaagggatttccctcctgaagggatttcccgagctcatc

SEQ ID NO: 35 - DNA, 63 bp, Artificial Sequence
  Primer VCAM 3 on page 34 second sequence
gatgagctcgggaaatcccttcaggagggaaatcccttcaggagggaaatccctggtaccgat

SEQ ID NO: 36 - DNA, 13 bp, Artificial Sequence
  ERE consensus sequence on page 35 line 3
ggtcannntgacc

SEQ ID NO: 37 - DNA, 409 bp, Artificial Sequence
  Cloned parts of rat oxytocin promotor on page 35 line 11-19
tacccgggatccaccgtcggtgatggtttctccagcccagaccgacctttttatgccttgtccactgccatggtggggcccagtctaagagggtgactgcatgactggtcacagccaggtctcttgggtcaaactgttccacactgtttagaagcaggcccttcatttgcagggtctgggctggggtcaaggtcaccgcctcagctaatgacctgagctcaaaagggacacagcctagaaggggaggcctaagctacaagaggataaagagacttggagggggtagaggtgcagcctagccaagagctgttttttcatagaaatccaatacctcagaatgaggttggatagcgcaagtgggtgaggaagcccttacgtggatctaaagctgacctgcaggcatgcaagc

SEQ ID NO: 38 - DNA, 13 bp, Artificial Sequence
  Well conserved ERE on page 35 line 13 in bold
ggtcaaactgttc

SEQ ID NO: 39 - DNA, 13 bp, Artificial Sequence
  Well conserved ERE on page 35 lines 14-15 in bold
ggtcaaggtcacc